(19) United States
(12) Patent Application Publication
(10) Pub. No.: US 2026/0054046 A1
(43) Pub. Date: Feb. 26, 2026

(54) DUAL-DEPLOYMENT SUTURE PLACEMENT & ANESTHESIA DELIVERY APPLIANCE AND RELATED DEVICES AND METHODS

(71) Applicant: New Wave Endo-Surgical Corp., Coconut Creek, FL (US)

(72) Inventors: Ricardo Alexander Gomez, Lighthouse Point, FL (US); Sandy Heck, Los Angeles, CA (US); Salvatore Castro, Coconut Creek, FL (US); William Strachan, Coconut Creek, FL (US); Miles Rosoff, Pompano Beach, FL (US); Oliva Kimmelman, Pompano Beach, FL (US); Lorelei Bennett, Pompano Beach, FL (US)

(21) Appl. No.: 18/815,818

(22) Filed: Aug. 26, 2024

(51) Int. Cl. A61M 39/02; A61B 17/04; A61M 19/00; A61M 25/02

(52) U.S. Cl. CPC A61M 39/0247; A61M 19/00; A61M 25/02; A61B 17/0482; A61M 2025/0286; A61M 2039/0205; A61M 2039/0261; A61M 2039/0291

(57) ABSTRACT

A suture placement device insertable into a surgical port has a cannular housing, and a suture guide mechanism movably connected to a distal housing portion arranged for movement between deployed and non-deployed positions. The mechanism defines first and second receptor port on opposite side portions and a suture passage therebetween that extends the housing distal end portion. First and second channel guides and the suture passage form the suture passage when deployed with portions of the first and second channel guides extending into corresponding receptor ports. Third and fourth channel guides forming injector channels are movable between retracted and deployed positions having portions extending from opposite sides to first and second injection positions. Actuation of a channel guide deployment mechanism attached to the housing and channel guides, such as slide, automatically deploys the first, second, third and fourth channel guides to their deployed positions allowing medicament injections and suture placement.

### CROSS-REFERENCE TO RELATED APPLICATIONS

This application is related to U.S. patent application Ser. No. 15/275,896 filed Sep. 26, 2016 at the U.S. Patent and Trademark Office (Now U.S. Pat. No. 9,986,987 to Patel et al. entitled “Apparatus and Method for Fascial Closure Device for Laparoscopic Trocar Port Site and Surgery”). Further, this application claims priority to U.S. Patent Application No. 63/407,315 filed Sep. 16, 20200. The disclosure of the above applications are incorporated herein by reference in their entirety.

### FIELD OF THE INVENTION

The present invention relates to fascial closure devices and related methods, and enhanced functionality of the same alone. More particularly, the present invention relates to fascial closure or suture placement arrangements having expanded functionality including anesthesia delivery functionality and/or suture placement in the deployed position, as well as methods for performing these and related functions.

### BACKGROUND

The subject matter described in the present application generally relates to laparoscopic surgery and closing abdominal wall defects. More particularly, the present application is directed to tissue closure devices including surgical suture devices as well as devices that can be used for intra-abdominal suturing, hernia repair, and closure of abdominal wall defects.

In the medical field, varying techniques for repairing damaged or diseased tissues are often used. In completing a surgical procedure various surgical methods employing sutures have been used to bind wounds, such as skin, muscles, tendons, internal organs, nerves, and blood vessels. Wound closure devices, such as sutures and staples as well as other repair devices like meshes or patches are used to repair and reinforce wounds.

With minimally invasive laparoscopic surgeries, the ability to tie surgical knots quickly and properly has presented a new challenge for surgeons. In cases in which knot tying is difficult, the use of knotless barbed suture wire can securely reapproximate tissues with less time, cost, and aggravation. The skills necessary to properly perform intra or extracorporeal knot tying for laparoscopic surgery can be achieved with practice and patience.

For discussion purposes and example representations to assist with describing aspects and features for embodiments herein, reference is made to laparoscopic surgical instruments described in U.S. Pat. No. 9,986,987 to Patel et al. (herein “The Patel Patent”). The Patel Patent describes various configurations of an apparatus and related methods for treating tissue openings, such as an endoscopic trocar port opening created and used for a minimally invasive surgical procedure, including a suture placement device configured to rapidly, safely, efficiently, and effectively close tissue defects created to access the intra-abdominal cavity during laparoscopic surgical procedures. The device as described can obtain adequate tissue adjacent to the tissue defect and provide a strong closure along with maintaining the pneumoperitoneum during the closure process, which includes techniques involving removal of the trocar from the port opening prior to placement of a fascial suture therein.

Despite the demonstrated effectiveness, reliability and other significant advantages proven by the suture placement device of the Patel Patent, surgical functionality and time-saving improvements can be implemented. In addition, continual challenges persist with respect to the duration of surgeries and the likelihood of complications increasing with duration, for which effective time-saving improvements and aids for improving efficiencies are consistently desired and pursued.

Referring now to FIGS. 1A to ID, a diagrammatic plan view of a minimally invasive surgical environment is shown that illustrates significant benefits and advantages that can be provided via the use of an effective suture placement device 50′ for port closure procedures. As shown in FIG. 1A, several small diameter ports are typically created through patient tissue for laparoscopic surgical procedures, such as intra-abdominal surgeries. These ports are often formed through the patient's skin 20′ (i.e., abdominal skin), fat layer 22′, through a fascial layer 24′, and sometimes muscles, which are each kept open as a laparoscopic port via use of a trocar port device 85′. The port depth A′ is determined and a trocar port device 85′ is selected to maintain the port during surgery along with a corresponding suture placement device 50′ for closing the same.

The trocar port device 85′ permits access for surgical instruments, laparoscopic cameras, and the like during surgery while simultaneously sealing the port to prevent the loss of inert gas. Inert gas, such as carbon dioxide, is typically pumped in through one or more ports to create a pneumoperitoneum space below the skin and above the surgical area to provide vital viewability for surgical procedures and maneuver space for surgical instruments and performing procedures. At the conclusion of the surgical procedures, these ports require effective suturing to close the corresponding wound and prevent herniation, which is best performed while maintaining the pneumoperitoneum and starting with effective placement of sutures at the fascial layer to close the port from the inside out.

Suture placement device 50′ greatly enhances the ability of a surgeon to effectively place sutures starting at the fascial layer 24′. The approach of effectively placing fascial sutures first and moving outward has been shown to enhance healing, reduce pain, and greatly reduce the possibility that the port will reopen. An effective method for placing sutures using a suture placement device 50′ is illustrated in FIGS. 1A to 1C, which includes inserting 62′ the distal end of an elongated cannula of the suture placement device 50′ through the corresponding port such that its distal end extends beyond the distal end of the trocar port device 85′, followed by withdrawing 64′ the trocar port device 85′ over the suture placement device 50′ as illustrated in FIG. 1A. The surgeon will select a suture placement device 50′ having a diameter slightly less than the inner diameter of the trocar port device 85′, such that suture placement device 50′ is able to maintain the port opening and prevent significant gas leakage until sutures are placed and the port is closed. The suture placement device 50′ is selected for the port such that its elongate cannula has a longitudinal length B′ sufficient for extending internally beyond the distal end of the trocar port device 85′, spanning the length of the port, and extending proximally an appropriate length for the surgeon to maintain control of the suture placement device and effectively use it for suture procedures.

The suture placement device 50′ is used to position a suture for closing intra-abdominal defects generated by surgical laparoscopic trocar ports and other puncturing devices, and to do so without any exposed sharps, which is enabled due to the suture placement device creating the suture path within the device and the suture being loaded therethrough. This is accomplished, in part, via rotation 66′ of a pivot bar or T-bar′ disposed at the distal end of the suture placement device 50′ about ninety degrees from its longitudinal orientation during insertion, such that the pivot bar is substantially parallel with the fascia layer 24′ and skin 20′, and extends across and beyond the width of the port as shown in FIG. 1B. Thereafter, the suture placement device 50′ is withdrawn 68′ externally until the top portions of the pivot bar are in contact with the fascia layer 24′.

Referring to FIG. 1C, stylet or channel guides 70′ disposed on opposite lateral sides of the cannula of the suture placement device 50′ are pushed downward or extended distally 70′ through the fascia (and muscle as appropriate) until each channel guide connects with and extends into corresponding openings formed in the rotated pivot bar. The elongate cannula of the suture placement device, the pair of stylet or channel guides, and the pivot bar each define channel segments therein. Upon connection of each stylet guide with and extending into the corresponding openings formed in the rotated pivot bar, the internal channel segments connect to form an uninterrupted internal channel pathway 80 within the suture placement device that extends through the fascia layer along a desired suture path.

As can be seen in FIG. 1D, the internal channel pathway defined through the suture placement device 50′ extends from an entry port 81′ formed at a proximal portion of the device longitudinally downward or distally within a first channel formed in the elongate cannula of the suture placement device to and through a first one of the stylet guides. The channel pathway 80′ continues uninterrupted around and through the rotated pivot bar at the distal end of suture placement device turning into and upwardly or proximally through the second one of the stylet guides. The channel pathway continues proximally through a second channel formed in the elongate cannula to an exit port 83′ formed at the proximal portion of the device. Thus, once suture placement device 50′ has been placed or installed within a port to be closed, and has been prepared for placement of a fascial suture or other suture, the suture placement device 50′ defines therethrough an uninterrupted channel pathway 80′ along a desired suture path.

After the suture thread has been directed through the channel pathway and extends along the pathway, the suture thread is in place to form a highly effective suture through the fascia layer 24′ for closing the defect. Once a suture thread is placed along the channel pathway, thin lateral slots along the stylet guides and pivot bar allow the suture thread to slide out of the channel pathway 80′ and the suture placement device 50′ while maintaining the desired placement through the fascia layer to establish the suture. The stylet guides can be withdrawn upward or proximally and the pivot bar can be rotated back to its initial elongate position to facilitate the suture thread withdrawing from the device while maintaining its suture position, as well as partial or complete proximal withdrawal of the suture placement device 50 out of the port as appropriate for releasing and completing the suture. Thereafter, a suture can be tightened and tied off to close the port at the fascia layer.

Referring to FIGS. 2A to 2C along with FIGS. 3A and 3B, a similar suture placement device 150′ is depicted in use for suture placement and port closure procedures through a trocar placement device 180′ as described above for Figures IA to ID with the exception of anesthetic delivery functionality as described hereafter. FIG. 3A depicts a similar example suture placement device 250′ in an anesthetic deployment arrangement similar to the arrangement shown in FIG. 2B for suture deployment device 150′, but with corresponding tissue removed for clarity purposes. Similarly, FIG. 3B depicts the example suture placement device 250′ in the suture placement arrangement shown in FIG. 2C for suture placement device 150′, but with tissue removed for clarity. As shown in FIG. 2A, the suture placement device 150′ is placed through the trocar placement device 180′ as described above for FIGS. 1A and 1B including rotation of the T-bar and its placement against the fascial plane. The T-bar or pivot bar serves as a fascial plane reference element and one or both stylet guides 170′ serves as an injection tube. A single guide plunger 140′ is used to set the depth of both stylet guides, such as via a fixed mechanical stop (not shown) or an adjustable mechanical stop (not shown) on the guide plunger.

Suture placement devices 150′ and 350′ include multiple modes or deployment positions including a deployment position for injecting anesthesia, and a separate deployment position for suture placement. FIGS. 2B and 3A depict an injection deployment mode showing the guide plunger 140′ partially depressed so that the tips of the stylet guides 170′ are positioned at a first distance H from the fascial plane reference element, which can be a beneficial position for injecting anesthesia prior to port closure. FIGS. 2C and 3B depict a suture placement mode showing the guide plunger fully depressed so that the stylet guide tips are fully deployed for suture placement similar to FIG. 1D described above. As such, the suture placement device 150′ is deployable for suture placement procedures through a two-step plunger deployment action, in which the first deployment action partially deploys the stylet guide tips for optional injection of anesthesia above the fascia plane and the second deployment action completes a suture placement pathway.

There is a need in the field for a Laparoscopic device capable of overcoming various drawbacks and challenges of conventional devices and significantly improving techniques, devices and related technology for closing defects along with injecting anesthesia proximate or above the fascia plane as desired without multiple device interactions or changing deployment modes. Further, there is a need for improved methods and techniques improved delivery and effectiveness of anesthesia on combination with port closure or suture placement procedures, as well as Laparoscopic devices for supporting the same.

### SUMMARY

One general aspect discussed herein along with example arrangement and method includes a suture placement device having a cannular housing insertable into a surgical port of a determined width. The housing can include: a proximal portion, a distal portion, and longitudinal axis extending therebetween; and a suture guide mechanism movably coupled to the distal portion of the cannular housing arranged for movement between a deployed position and a non-deployed position. The suture guide mechanism defines a first receptor port, a second receptor port, and a suture passage therebetween. The first and second receptor ports are disposed on opposite side portions of the cannular housing and the suture guide mechanism in the deployed position, and the suture passage extends across a width of the cannular housing at a distal end in the deployed position. The device also includes a first channel guide within the housing that can include a proximal end, a distal end, and a guided suture path segment therebetween, in which the first channel guide is movable between a retracted position oriented for passage through the surgical port and a deployed position where a portion of the first channel guide extends from the housing to the first receptor port. The device also includes a second channel guide within the housing that can include a proximal end, a distal end, and a guided suture path segment therebetween, in which the second channel guide is movable between a retracted position oriented for passage through the surgical port and a deployed position where a portion of the second channel guide extends from the housing to the second receptor port. The device further includes a third channel guide within the housing that can include a proximal end, a distal end, and an injection path segment therebetween, in which the third channel guide is movable between a retracted position oriented for passage through the surgical port and a deployed position where a portion of the third channel guide extends from a first side of the housing to a first injection position. The device also includes a fourth channel guide within the housing that can include a proximal end, a distal end, and an injection path segment therebetween, in which the fourth channel guide is movable between a retracted position oriented for passage through the surgical port and a deployed position where a portion of the fourth channel guide extends from a second side of the housing opposite from the first side to a second injection position. In addition, the device includes a channel guide deployment mechanism attached to the housing and each channel guide. Actuation of the channel guide deployment mechanism simultaneously deploys the first, second, third and fourth channel guides to each of the respective deployed positions.

Implementations can include one or more of the following features. Actuation of the channel guide deployment mechanism can simultaneously place the suture placement device in condition for suturing and for injecting a medicament through the third and fourth channel guides. In the actuated and deployed configuration, each of the first, second, third and fourth channel guides can be in the respective deployed position, such that: A continuous suture pathway is defined through the suture placement device from a first opening at the proximal portion of the housing through each of the first and second channel guides and the suture guide mechanism therebetween to a second opening at the proximal portion of the housing; A first injection pathway is defined through the suture placement device from a third opening at the proximal portion of the housing through the third channel guide to the distal end of the third stylet guide at the first injection position; and A second injection pathway is defined through the suture placement device from a fourth opening at the proximal portion of the housing, through the fourth channel guide to the distal end of the fourth channel guide at the second injection position.

The first opening at the proximal portion of the housing and the second opening at the proximal portion of the housing can be formed through a top portion of the housing. The first and second openings can be axially aligned with each other across a first radial axis of the housing top portion. The third opening at the proximal portion of the housing and the fourth opening at the proximal portion of the housing can also be formed through a top portion of the housing. In some implementations, the third and the fourth openings can be axially aligned with each other across a second radial axis of the housing top portion. In some arrangements, the first radial axis and the second radial axis can generally be aligned with each other such that the first, second, third and fourth openings are generally radially aligned along the same radial axis across the housing top portion. In some arrangements, the first radial axis can define an acute angle with the second radial axis.

In some implementations, the first and second openings can be defined through a top surface of the top portion of the housing; and the third and fourth openings can be defined through proximal side portions of the housing at the top portion of the housing. The suture placement device can include an injection connector configured for coupling each of the third and fourth openings with a top portion of a medicament injector. The first and second channel guides can each extend a first depth in the surgical port when in the respective deployed positions, the third channel guide can extend a second depth in the surgical port when in the deployed position at the first injection position, and the fourth channel guide can extend a third depth in the surgical port when in the deployed position at the second injection position. In some arrangements, the second and third depths are less than the first depth. In some arrangements, the first and the second injection positions and the second and third depths can be substantially the same.

In the actuated and deployed configuration when each of the first, second, third and fourth stylet guides are in the respective deployed position, the first channel guide and the third channel guide can be disposed proximate each other extending from a first side of the housing, and the second channel guide and the fourth channel guide can be disposed proximate each other extending from a second side of the housing opposite from the first side of the housing. In the actuated and deployed configuration in which each of the first, second, third and fourth channel guides are in the respective deployed position, the first channel guide can extend from a third side of the housing, the second channel guide can extend from a fourth side of the housing opposite from the third side of the housing, and the first and second channel guides can define a first cross-sectional axis therebetween. The third channel guide and the fourth channel guide can further define a second cross-sectional axis therebetween; and an acute angle can be defined between the first and the second cross-sectional axes. The third channel guide and the fourth channel guide can be oriented substantially perpendicular to the first channel guide and the second channel guide, and the acute angle can be about ninety degrees.

In some implementations, a portion of the injection path segment of each of the third and fourth channel guides can be fenestrated. Further, the distal end of each of the third and fourth channel guides can be substantially closed. In some implementations, the fenestrated portion of each injection path segment can be a distal end portion of the injection path segment, and a substantial portion of a deployed distal length of each injection path segment that extends from the housing can be fenestrated. Further, the third channel guide and the fourth channel guide can be formed from flexible tubes.

A method of use for a suture device according to examples described herein can include placing a suture and injecting a medicament in tissue through a surgical port therein having an outer diameter. The method can include inserting the suture placement device into the surgical port, moving the suture guide mechanism into the deployed position, and actuating the channel guide deployment mechanism for simultaneously deploying the first, second, third and fourth channel guides to the deployed positions. Once deployed, the method can further include injecting a first medicament into an entry, such as an injector port of a first injector pathway connected with the third channel guide for injecting the first medicament into the tissue at the first side of the housing. In the deployed condition, the method can further include injecting a second medicament into an entry, such as an injector port, into a second injector pathway connected with the fourth channel guide for injecting the second medicament into the tissue at the second side of the housing. In some implementations, a medicament can be simultaneously injected into both the first and the second injector pathways via an injection adapter coupled to input ports for the third and fourth channel pathways. The method can further include routing a suture through a suture path formed through the housing, the first channel guide, the second channel guide and the suture passage of the suture guide mechanism, removing the suture placement device while keeping the suture in place, and tying the suture.

In some methods, for injecting the first medicament, the first medicament can include at least one of an anaesthesia, a nsaid, and a therapeutic. Similarly, for injecting the second medicament, the second medicament can include at least one of an anaesthesia, a nsaid, and therapeutic. In some methods, for actuating the channel guide deployment mechanism, actuating the channel guide deployment mechanism can simultaneously deploy the first, second, third and fourth channel guides to the deployed positions. In some methods, for actuating the channel guide deployment mechanism, actuating the channel guide deployment mechanism can implement deployment of the first, second, third and fourth channel guides to the deployed positions in a sequential manner and/or combination of sequential and simultaneous, such as deploying a first pair of the channel guides followed by automatically deploying a second pair of the channel guides. Further, the method can include: providing an abdominal wall nerve block by deploying the third and the fourth channel guides to the deployed positions at the first and second injection positions spaced at desired distances of about 0.1 to 1.0 cm proximal from the first and second receptor ports, respectively, and injecting an anaesthetic into the third and fourth channel guides.

Other aspects and advantages according to aspect, features, innovative concepts and implementations discussed along with example devices, arrangements and methods pertaining to the present invention will become apparent from the following detailed description, which when taken in conjunction with the accompanying drawings illustrates by way of example principles of the invention.

### BRIEF DESCRIPTION OF THE DRAWINGS

To further clarify the above and other advantages and features of the present invention, a more particular description of the invention will be rendered by reference to specific embodiments thereof which are illustrated in the appended drawings. It is appreciated that these drawings depict only illustrated embodiments of the invention and are therefore not to be considered limiting of its scope. The invention will be described and explained with additional specificity and detail through the use of the accompanying drawings in which:

FIG. 1A is a diagrammatic plan view of a minimally invasive surgical environment configured to use an example prior art surgical device in the form of a suture placement device shown in a diagrammatic surgical environment useful for understanding aspects and features of the subject matter described herein, which as shown includes a prior art trocar device installed through a laparoscopic surgery port formed through patient tissue along with the example prior art suture placement device inserted therethrough in preparation for port closure procedures.

FIGS. 1B and 1C are plan views of the prior art example suture placement device of FIG. 1A in the environment of FIG. 1A showing example actions pertaining to arranging the suture placement device of FIG. 1A for placement of a suture, for which aspects and features discussed herein are related.

FIG. 1D is a plan view of the suture placement device and environment of FIGS. 1A-IC showing an arrangement of the suture placement device prepared for placement of a suture prior to introduction of a guide wire including having a channel pathway established through the device.

FIG. 2A is a schematic plan view of another prior art suture placement device that depicts entry of the example prior art device into an optional trocar device and movements of a suture channel assembly from a first arrangement to a second arrangement.

FIG. 2B is a schematic plan view of the example prior art suture placement device of FIG. 2A schematically depicting an injection deployment position for injection of an analgesic into a pre-peritoneum area after removing the trocar.

FIG. 2C is a schematic plan view of the example prior art suture placement device of FIGS. 2A and 2B depicting a suture placement deployment position for routing and placing suture material about the surgical port for port closure actions.

FIG. 3A is a schematic plan view of an example prior art suture placement device corresponding with the device of FIGS. 2A and 2B showing an example injection deployment position corresponding with FIG. 2B for injection of an analgesic into a pre-peritoneum area after removing the trocar, which is shown with the corresponding tissue removed for clarity purposes.

FIG. 3B is a schematic plan view of the example prior art suture placement device corresponding with the device of FIGS. 2A-2C showing an example suture placement deployment position corresponding with FIG. 3C with the corresponding tissue removed for clarity purposes.

FIG. 4A is a schematic plan view of an example dual-deployment suture placement and injection device according to aspects and features described herein having a deployment position for allowing injection of one or more medicaments through deployed injection channels and/or suture placement actions through a deployed suture pathway extending from a first proximal input port to a second proximal input port.

FIG. 4B is a proximal end view of the example dual-deployment suture placement and injection device of FIG. 4A as indicated in FIG. 4A.

FIG. 4C is a distal end view of the example dual-deployment suture placement and injection device of FIG. 4A as indicated in FIG. 4A taken at a distal position located proximally just past the bullet tip suture pathway.

FIG. 5A is a schematic plan view of a distal end portion of another example dual-deployment suture placement and injection device according to aspects and features described herein, which is similar to FIG. 4A also has a combined functionality deployment position according to another example arrangement.

FIG. 5B is a perspective view of the distal end portion of the device of FIG. 5A, and FIG. 5C is close perspective view of a distal end of the device of FIGS. 5A and 5B.

FIG. 6A is an exploded view of the proximal end portion of the device of FIG. 5A showing an injector connection device, an upper portion of the cannula, a pair of suture channel guides along opposite side portions of the device, and a pair of injection channel guides along opposite side portion of the device arranged, in which the each pair member along a corresponding pair portion are arranged parallel and adjacent to one another.

FIG. 6B is an exploded perspective view of the channel guide members and upper cannula portion of FIG. 6A.

FIG. 7 is a perspective view of the injector connection device of FIG. 6A.

FIG. 8A is a perspective view of upper regions of the upper portion of the cannula and the pairs of channel guides shown in FIG. 6A, which are depicted along with FIG. 8B showing the injector connector device of FIG. 6A along with dashed lines showing a connection arrangement between the injector connector device and the pair of injector channels.

FIG. 9A is a proximal end view of the dual-deployment suture placement and injection device of FIGS. 5A and 6A as shown in FIG. 6A.

FIG. 9B is a distal end view of the dual-deployment suture placement and injection device of FIG. 5A from the position shown in FIG. 5A located proximally of the bullet tip end portion of the device.

FIG. 10A is a perspective view of a proximal end portion of an additional dual-deployment suture placement and injection device according to aspects and features described herein, which depicts a pair of suture channel entry ports aligned with each other along a first radial axis across the device on opposite sides of the housing, and a pair of injection channel entry ports aligned with each other along a second radial axis across the device on opposite sides of the housing, in which the first and second radial axes are oriented normal to each other.

FIG. 10B is a proximal end view of the portion of a dual-deployment suture placement and injection device shown in FIG. 10A, which depicts a rotation angle, (2, between the first and second radial axes.

FIG. 11 is a perspective view of a further schematic example dual-deployment suture placement and injection device according to aspects and features described herein, which depicts injection channel guides radially rotated with respect suture channel guides in an example offset arrangement at a proximal end portion, and a different radially rotated offset arrangement at a radially rotated offset arrangement at a distal end portion, which is shown for example purposes regarding flexibility for channel guide orientations.

FIG. 12 is a close perspective view of a distal end portion of the example dual-deployment suture placement and injection device of FIG. 11.

FIG. 13A is a proximal end view of the example dual-deployment suture placement and injection device of FIG. 11 as indicated in FIG. 11, and FIG. 13B is a distal end view thereof as also indicated in FIG. 11.

FIG. 14 is a close perspective view of yet another schematic example dual-deployment suture placement and injection device according to aspects and features described herein, which has a distal end proton arrangement corresponding with the distal end portion arrangement of FIG. 11 except for the use of partially fenestrated injection channel guide.

FIG. 15A is a close perspective view of a distal end portion of a suture channel guide and of an injection channel guide of the device of FIG. 14 for the view indicated on FIG. 14.

FIG. 15B is close perspective view corresponding with the FIG. 15 showing an alternative arrangement for a distal end portion of a partially fenestrated injection channel guide according to aspects and features described herein.

FIG. 16 is a schematic representation of an example method for using a dual-deployment suture placement and injection device according to aspects and features described herein.

### DETAILED DESCRIPTION OF THE DRAWINGS

While the various features of this invention are hereinafter described and illustrated as being particularly adapted for providing rigidity to instrument devices the invention is not limited to the embodiments illustrated in the drawings but are merely used to illustrate the wide variety of uses of this invention. Therefore, the foregoing is considered as illustrative only of the principles of the invention. Since numerous modification and changes will readily occur by those skilled in the art, it is not desired to limit the invention to the exact construction and operation shown and described and accordingly, all suitable modifications and equivalents may be resorted to, falling within the scope of the invention.

Aspects, features and concepts described herein pertaining to surgical apparatus, methods, and devices are directed to apparatus and methods for closing tissue opening including puncture wounds, hernias, endoscopic trocar port openings used in minimally invasive surgical procedures, and other abdominal wall defects in the fascia. Aspects, concepts and features pertaining to a dual-deployment suture placement and injection device are discussed for rapidly, safely, efficiently and effectively repairing hernias and other tissue defects in a human body.

Example dual-deployment suture placement and injection device as described herein can be able to obtain adequate tissue adjacent to the tissue defect to provide a strong closure, to maintain pneumoperitoneum needed for appropriate visualization of the peritoneal contents during the closure process, and to protect the vital structures within the abdominal cavity in the vicinity and the healthcare provider for risk of injury, as well as allow injection of one or more medicaments while the device is deployed for suture placement procedures.

There is currently no single device that provides means similar to examples discussed herein for performing fascial port closure along with medicament injection, such as anesthesia injections. These and other novel and innovative concepts and improvements discussed herein can be performed using a single device according to aspects and features described herein along with example arrangements, which can include for example applying a nerve block with the same device in a common deployment arrangement for performing fascial port closure. Other enhancements, concepts and features described herein can include implementation of significantly improved closures, which can be further improved by adhering to Johnsson-Israelson rules and guidelines for fascial closure, such as implement symmetrical and evenly spaced suture bites equidistant from a center of a defect, such as 1 cm along with the application of local anesthesia above the peritoneum within the defect.

As used herein, the term “about” when used in connection with a referenced numeric indication means the referenced numeric indication plus or minus up to 10 percent of that referenced numeric indication. For example, the language “about 50” covers the range of 45 to 55. Similarly, the language “about 5” covers the range of 4.5 to 5.5.

As used herein, the term “target workspace” refers to anything within or pertaining to the endoscopic work cavity including the body of the patient, tissues and organs within the cavity, and tissue defining the cavity, and also to support structures for the MIS procedure including a cover and cannula supports, instruments and related attachments or medical implements including needles, suture materials, implants, meshes, etc. As used herein, the term “target tissue” refers to any tissue or organ that interacts with the target workspace including tissues and organs of the patient, natural tissues and organs introduced to the target workspace including natural transplant tissues and organs, artificial tissues and organs including mechanical or electro-mechanical organs, and tissue and organ assist devices such as pacemakers, mesh material, artificial skin and the like.

As used herein, a surgical device or tool or clinical instrument refers to a medical instrument having contact surfaces that are configured to engage organs, tissues and/or portions of a surgical cavity or wound to thereby move, hold, lift, retain, suture or otherwise engage, interface or make contact with the target tissue and perform clinical functions as appropriate for the surgical environment. The term “flexible” in association with a part, such as a mechanical structure, component, or component assembly, should be broadly construed. In essence, the term means the part can be repeatedly bent and restored to an original shape without harm to the part. Certain flexible components can also be resilient. For example, a component (e.g., a flexure) is said to be resilient if possesses the ability to absorb energy when it is deformed elastically, and then release the stored energy upon unloading (i.e., returning to its original state). Many “rigid” objects have a slight inherent resilient “bendiness” due to material properties, although such objects are not considered “flexible” as the term is used herein.

A flexible part may have infinite degrees of freedom (DOF's). Flexibility is an extensive property of the object being described, and thus is dependent upon the material from which the object is formed as well as certain physical characteristics of the object (e.g., cross-sectional shape, length, boundary conditions, etc.). For example, the flexibility of an object can be increased or decreased by selectively including in the object a material having a desired modulus of elasticity, flexural modulus, and/or hardness. The modulus of elasticity is an intensive property of (i.e., is intrinsic to) the constituent material and describes an object's tendency to elastically (i.e., non-permanently) deform in response to an applied force. A material having a high modulus of elasticity will not deflect as much as a material having a low modulus of elasticity in the presence of an equally applied stress. Thus, the flexibility of the object can be decreased, for example, by introducing into the object and/or constructing the object of a material having a relatively high modulus of elasticity.

As used in this specification and the appended claims, the word “distal” refers to direction towards a work site, and the word “proximal” refers to a direction away from the work site. Thus, for example, the end of a tool that is closest to the target tissue would be the distal end of the tool, and the end opposite the distal end (i.e., the end manipulated by the user or coupled to an actuation shaft) would be the proximal end of the tool.

Further, specific words chosen to describe one or more embodiments and optional elements or features are not intended to limit the invention. For example, spatially relative terms such as “beneath”, “below”, “lower”, “above”, “upper”, “proximal”, “distal”, and the like—may be used to describe the relationship of one element or feature to another element or feature as illustrated in the figures. These spatially relative terms are intended to encompass different positions (i.e., translational placements) and orientations (i.e., rotational placements) of a device in use or operation in addition to the position and orientation shown in the figures. For example, if a device in the figures were turned over, elements described as “below” or “beneath” other elements or features would then be “above” or “over” the other elements or features. Thus, the term “below” can encompass both positions and orientations of above and below. A device may be otherwise oriented (e.g., rotated 90 degrees or at other orientations) and the spatially relative descriptors used herein interpreted accordingly. Likewise, descriptions of movement along (translation) and around (rotation) various axes includes various spatial device positions and orientations. The combination of a body's position and orientation define the body's pose.

Similarly, geometric terms, such as “parallel”, “perpendicular”, “round”, or “square”, are not intended to require absolute mathematical precision, unless the context indicates otherwise. Instead, such geometric terms allow for variations due to manufacturing or equivalent functions. For example, if an element is described as “round” or “generally round.” a component that is not precisely circular (e.g., one that is slightly oblong or is a many-sided polygon) is still encompassed by this description.

In addition, the singular forms “a”, “an”, and “the” are intended to include the plural forms as well, unless the context indicates otherwise. The terms “comprises”, “includes”, “has”, and the like specify the presence of stated features, steps, operations, elements, components, etc. but do not preclude the presence or addition of one or more other features, steps, operations, elements, components, or groups.

Unless indicated otherwise, the terms apparatus, medical device, instrument, and variants thereof, can be interchangeably used.

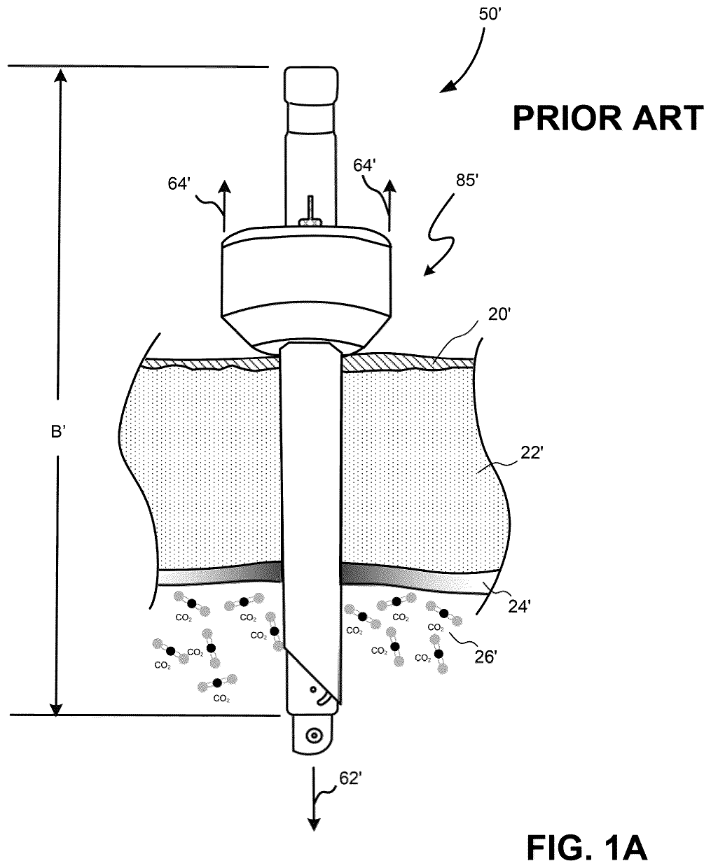

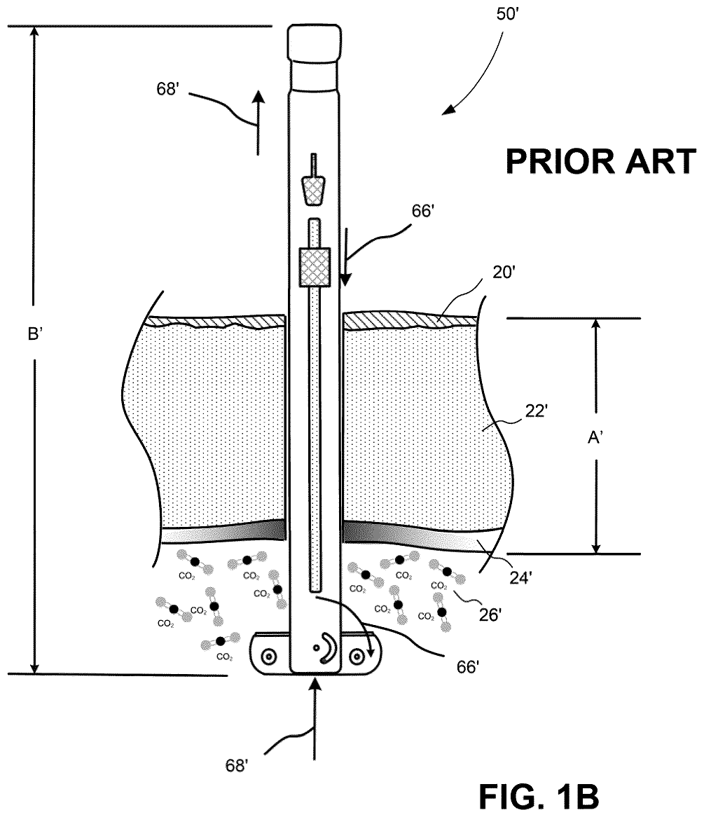

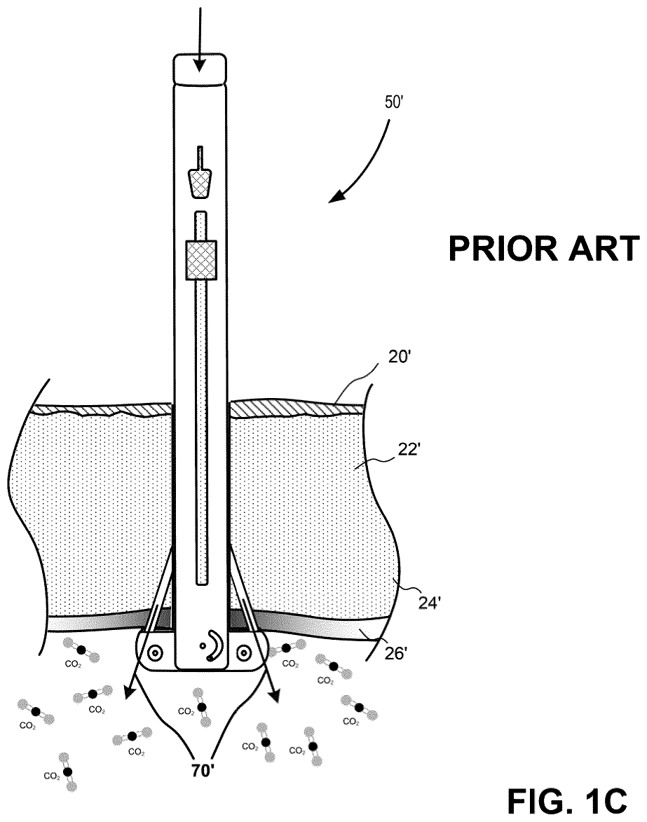

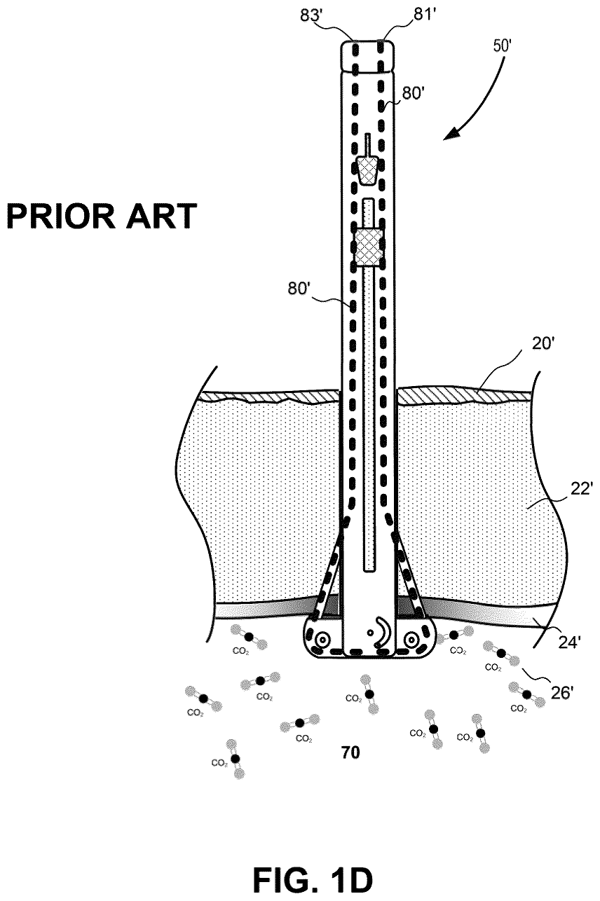

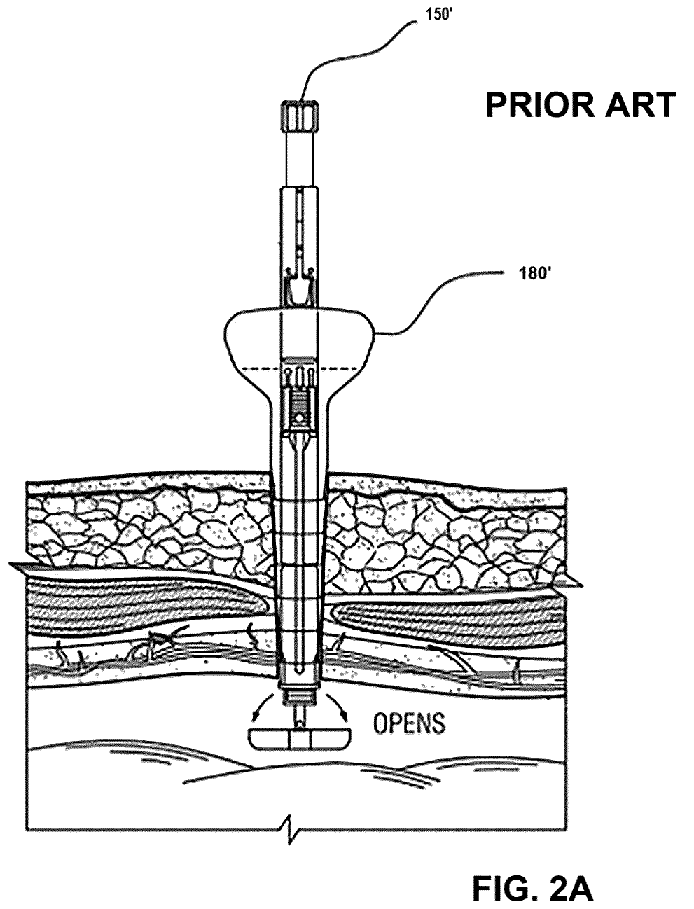

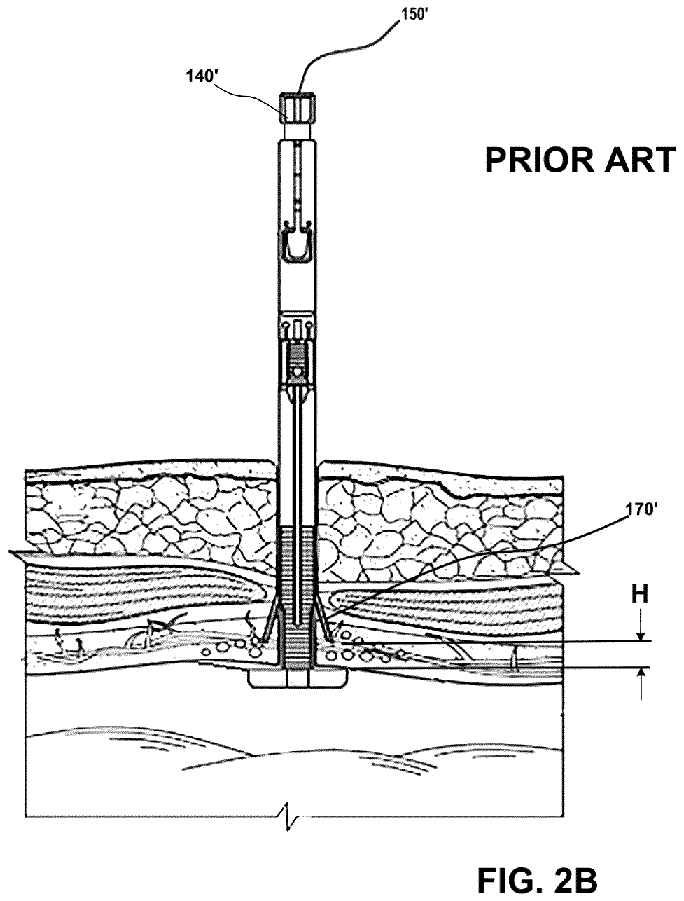

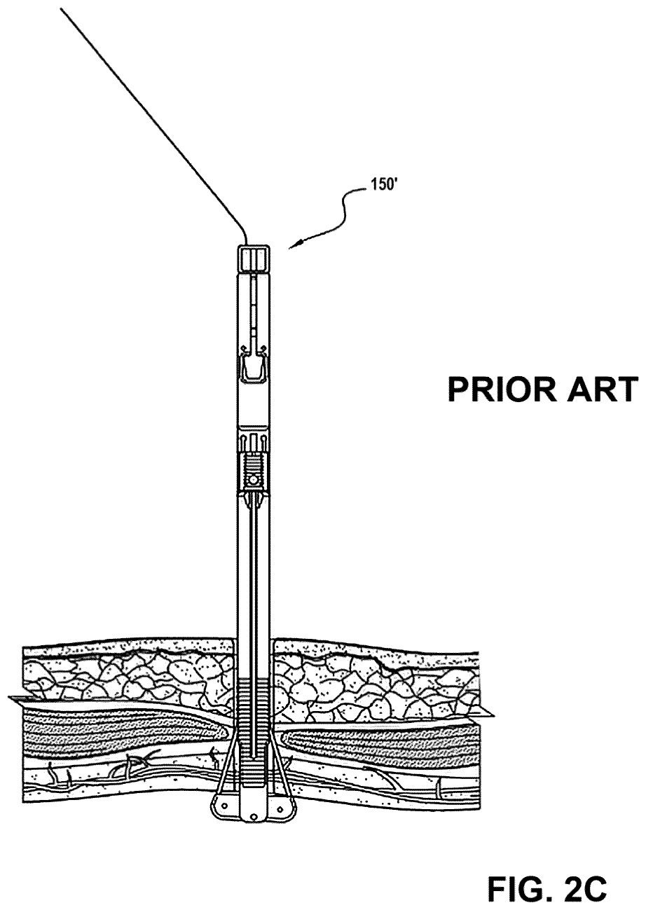

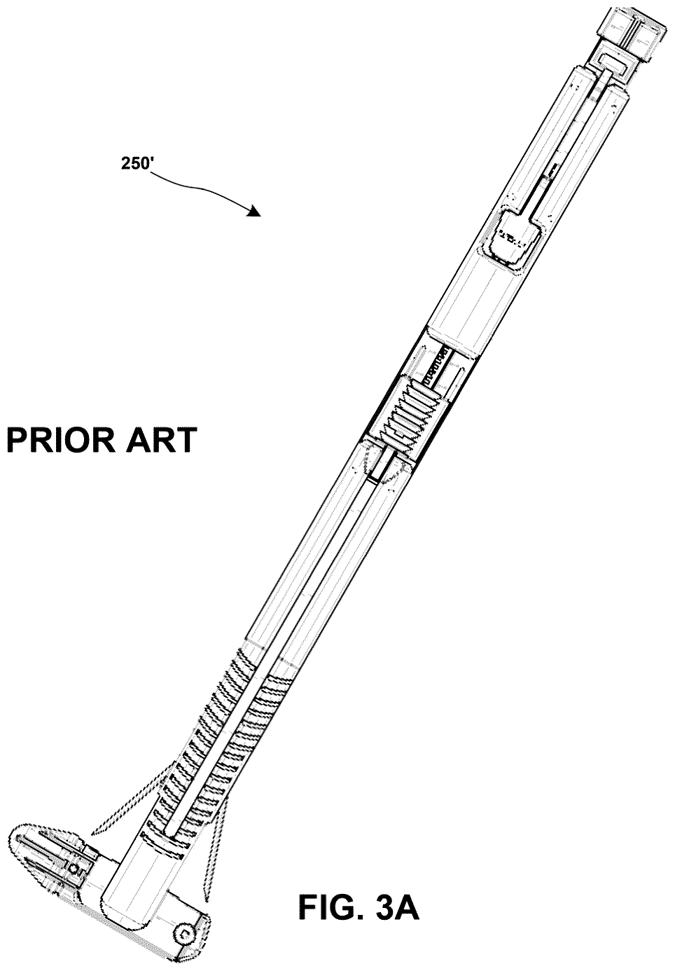

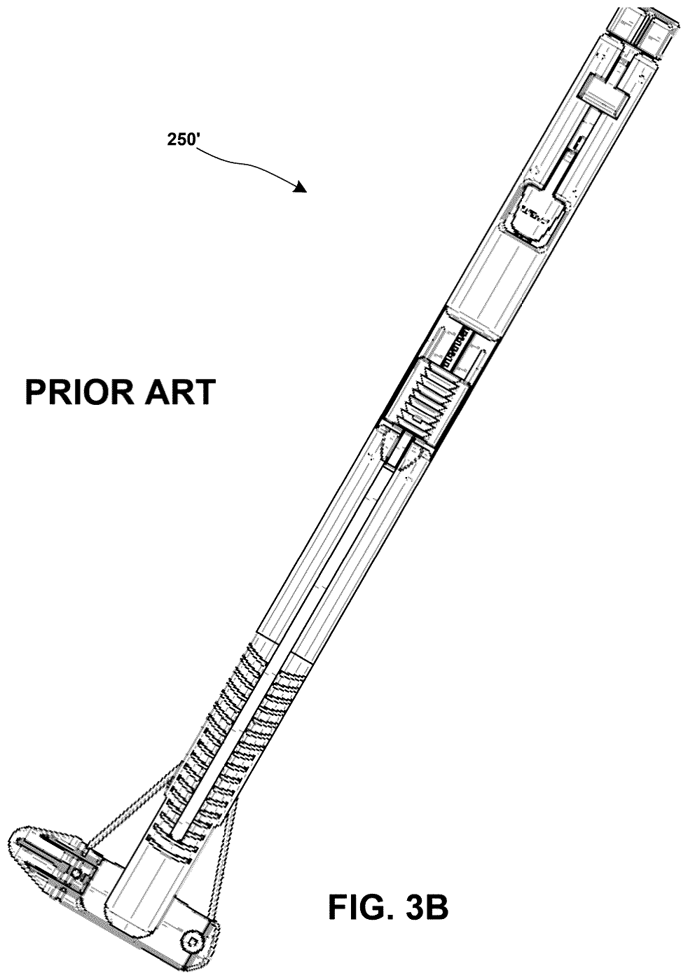

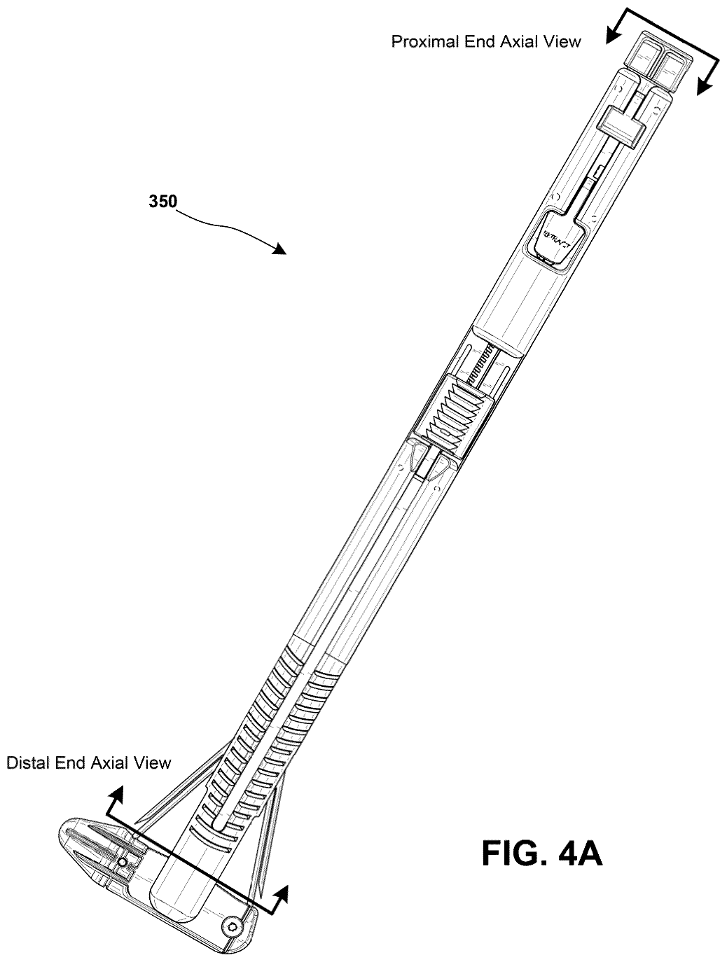

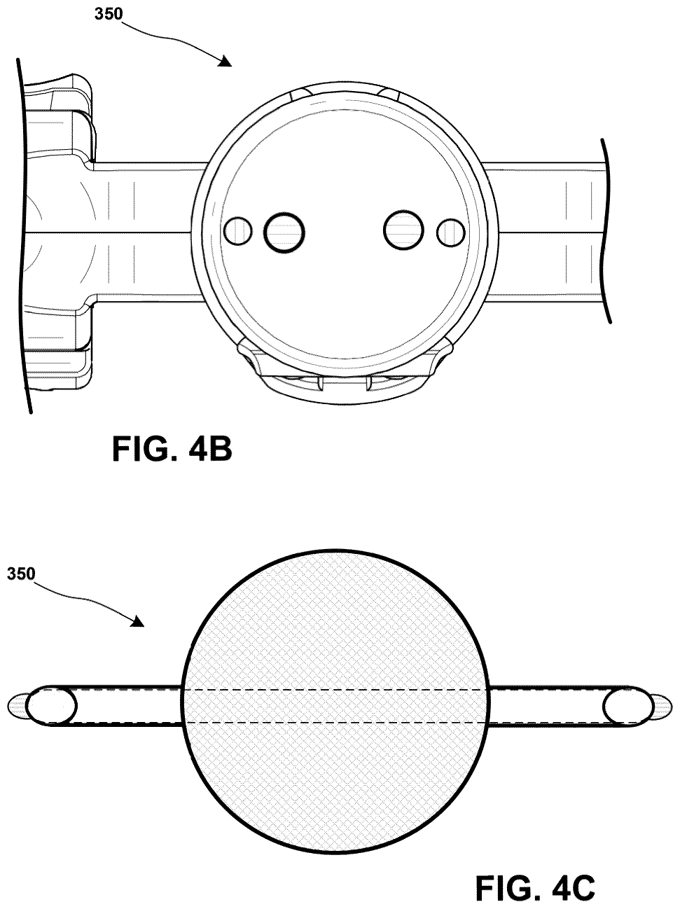

Referring now to FIGS. 4A and 1B, a dual-deployment suture placement and injection device is shown having a cannular housing insertable into a surgical port of a determined width. The housing can include: a proximal portion, a distal portion, and longitudinal axis extending therebetween; and a suture guide mechanism movably coupled to the distal portion of the cannular housing arranged for movement between a deployed position and a non-deployed position. The suture guide mechanism defines a first receptor port, a second receptor port, and a suture passage therebetween. The first and second receptor ports are disposed on opposite side portions of the cannular housing in the deployed position and the suture passage extends across a width of the cannular housing in the deployed position. The device also includes a first channel guide within the housing that can include a proximal end, a distal end, and a guided suture path segment therebetween, in which the first channel guide is movable between a retracted position oriented for passage through the surgical port and a deployed position where a portion of the first channel guide extends from the housing to the first receptor port. The device also includes a second channel guide within the housing that can include a proximal end, a distal end, and a guided suture path segment therebetween, in which the second channel guide is movable between a retracted position oriented for passage through the surgical port and a deployed position where a portion of the second channel guide extends from the housing to the second receptor port. The device further includes a third channel guide within the housing that can include a proximal end, a distal end, and an injection path segment therebetween, in which the third channel guide is movable between a retracted position oriented for passage through the surgical port and a deployed position where a portion of the third channel guide extends from a first side of the housing to a first injection position. The device also includes a fourth channel guide within the housing that can include a proximal end, a distal end, and an injection path segment therebetween, in which the fourth channel guide is movable between a retracted position oriented for passage through the surgical port and a deployed position where a portion of the fourth channel guide extends from a second side of the housing opposite from the first side to a second injection position. In addition, the device includes a channel guide deployment mechanism attached to the housing and each channel guide. Actuation of the channel guide deployment mechanism simultaneously deploys the first, second, third and fourth channel guides to each of the respective deployed positions.

Actuation of the channel guide deployment mechanism can simultaneously place the dual-deployment suture placement and injection device in condition for suturing and for injecting a medicament through the third and fourth channel guides. In the actuated and deployed configuration, each of the first, second, third and fourth channel guides can be in the respective deployed position, such that: A continuous suture pathway is defined through the dual-deployment suture placement and injection device from a first opening at the proximal portion of the housing through each of the first and second channel guides and the suture guide mechanism therebetween to a second opening at the proximal portion of the housing; A first injection pathway is defined through the dual-deployment suture placement and injection device from a third opening at the proximal portion of the housing through the third channel guide to the distal end of the third stylet guide at the first injection position; and A second injection pathway is defined through the dual-deployment suture placement and injection device from a fourth opening at the proximal portion of the housing, through the fourth channel guide to the distal end of the fourth channel guide at the second injection position.

The first opening at the proximal portion of the housing and the second opening at the proximal portion of the housing can be formed through a top portion of the housing. The first and second openings can be axially aligned with each other across a first radial axis of the housing top portion. The third opening at the proximal portion of the housing and the fourth opening at the proximal portion of the housing can also be formed through a top portion of the housing. As shown in FIG. 4B, the third and the fourth openings can be axially aligned with each other across a second radial axis of the housing top portion.

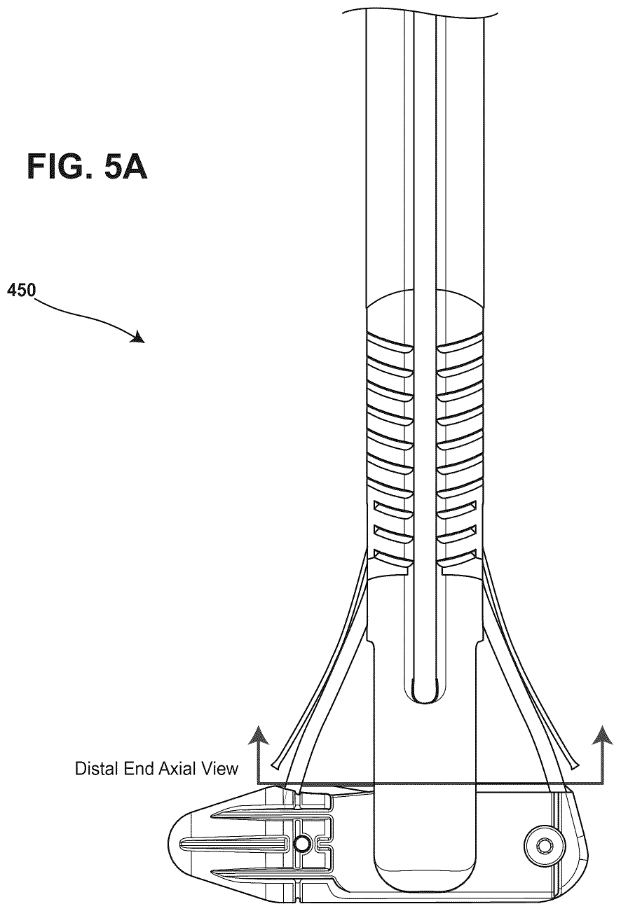

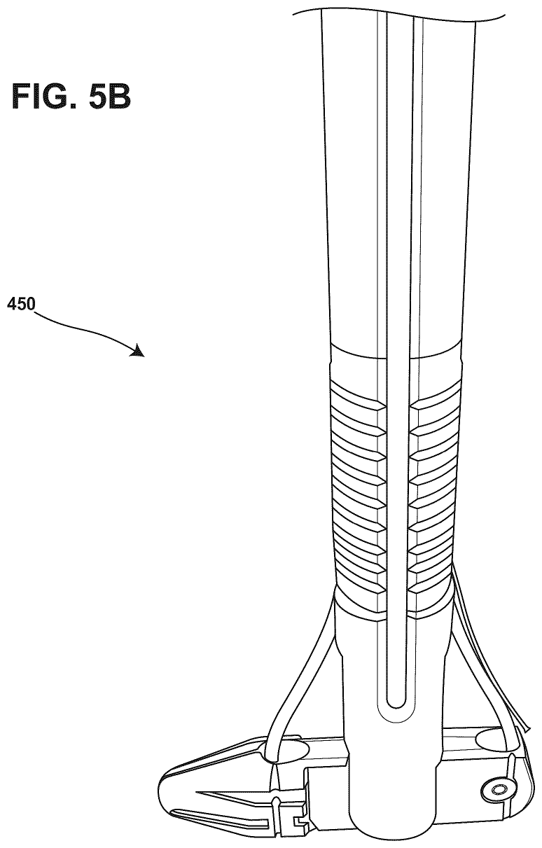

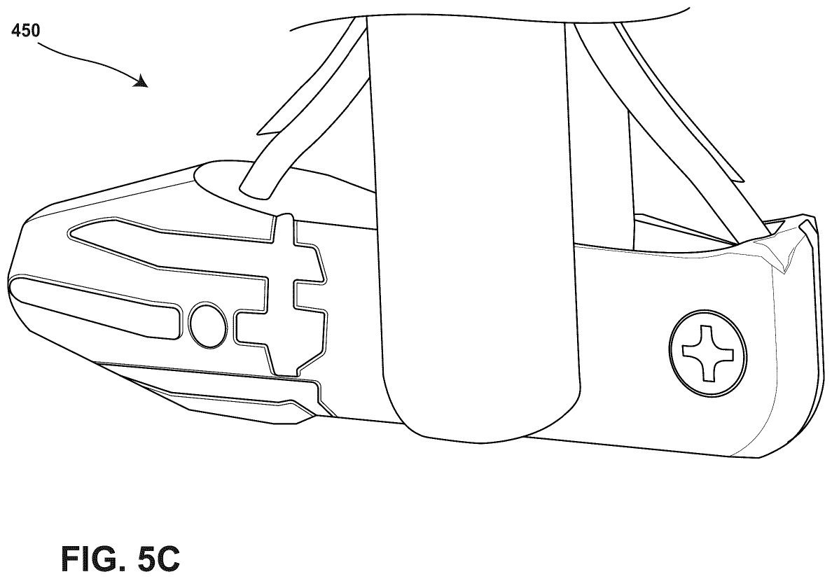

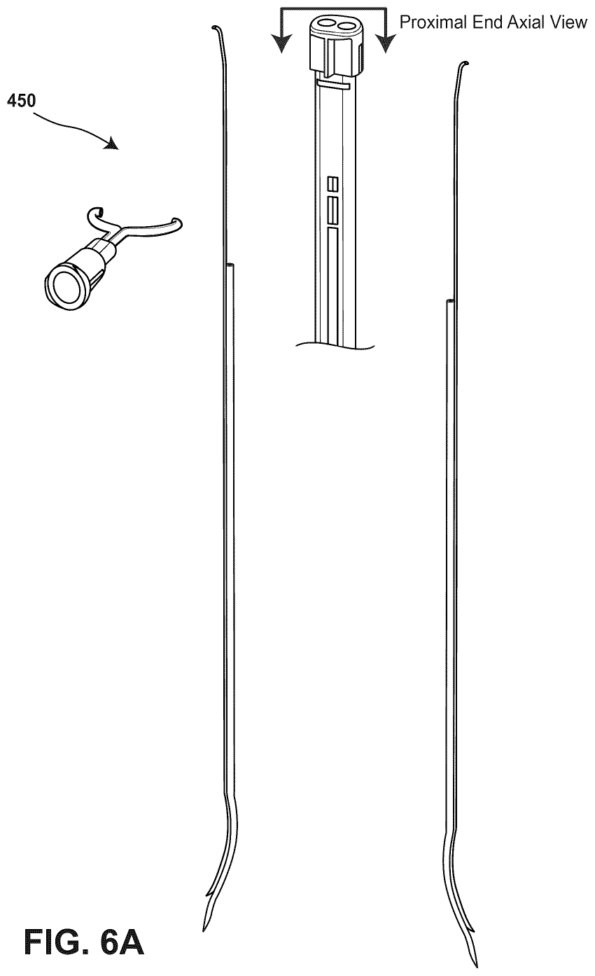

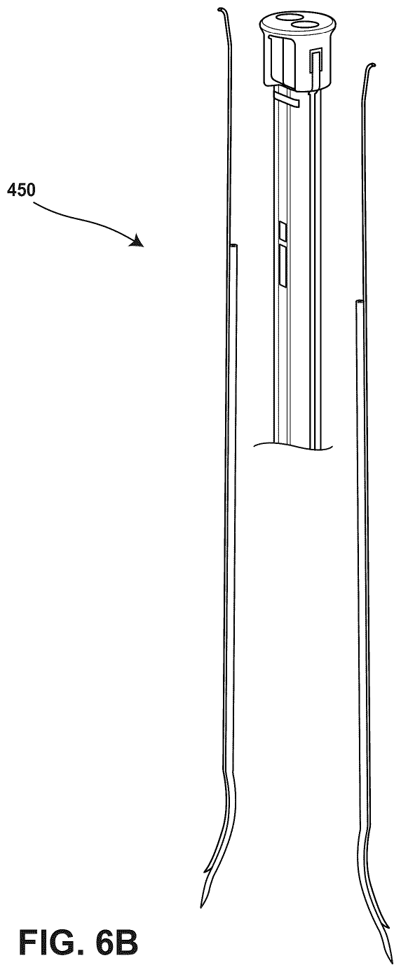

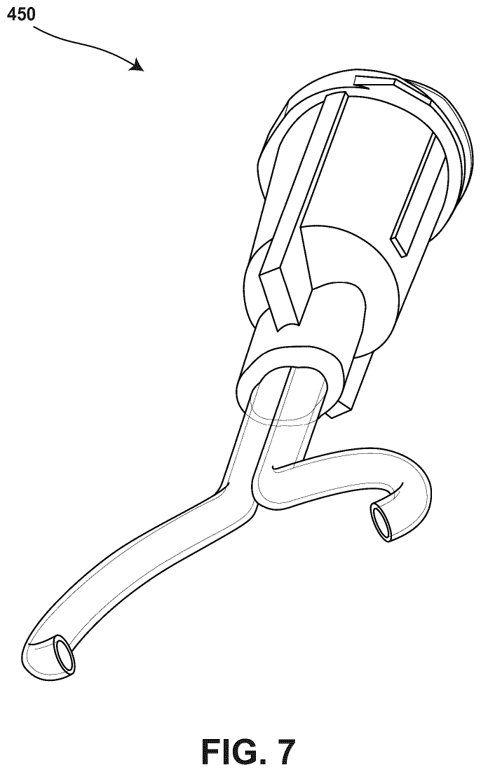

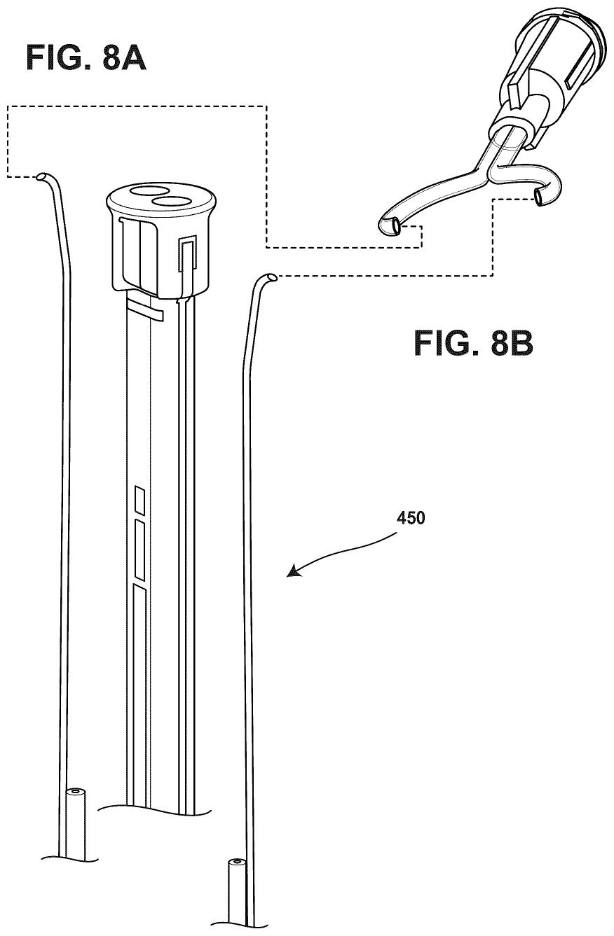

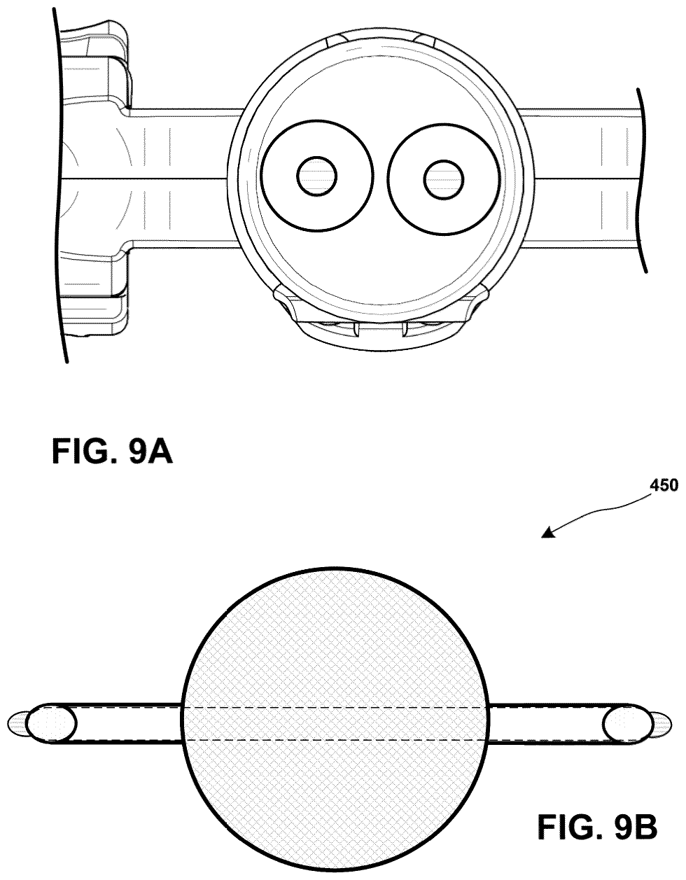

Referring now to FIGS. 5A to 9B, another schematic example of a dual-deployment suture placement and injection device is shown that generally has the same aspects and features as the device of FIGS. 4A and 4B, except as shown and described herein. In some arrangements including the arrangement of FIGS. 5A to 9B and also as shown for the device of FIGS. 4A and 4B, the first radial axis and the second radial axis between input ports for the injector channel guides and the suture channel guides formed in a proximal end portion of the device can generally be aligned with each other such that the first, second, third and fourth openings are generally radially aligned along the same radial axis across the housing top portion. The input ports can be radially aligned at the proximal end portion, as well as the channel guides, such that an injector channel guide and a suture channel guide on each side of the housing can be oriented and arranged parallel and adjacent to one another extending along the length of the device as depicted for the device of FIGS. 4A to 9B. Further, in some arrangements as depicted for the present example, injector input ports can be defined through proximal side portions of the housing at the top portion of the housing. Further, a connector device can facilitate injection of a medicament into input ports for the injectors channel guides and thereby enable a tip of a single injection device, such as a needle (not shown) to inject medicament through the pair of injector channel guides simultaneously.

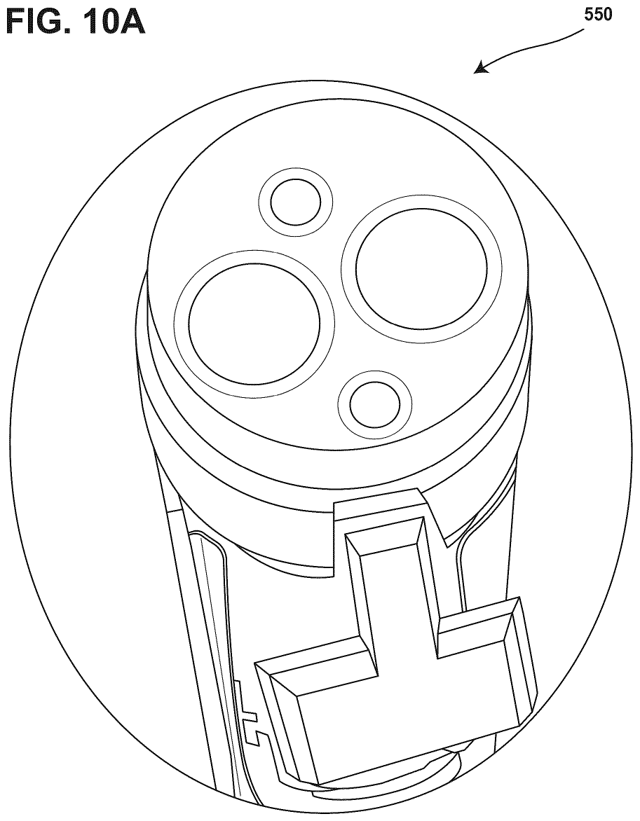

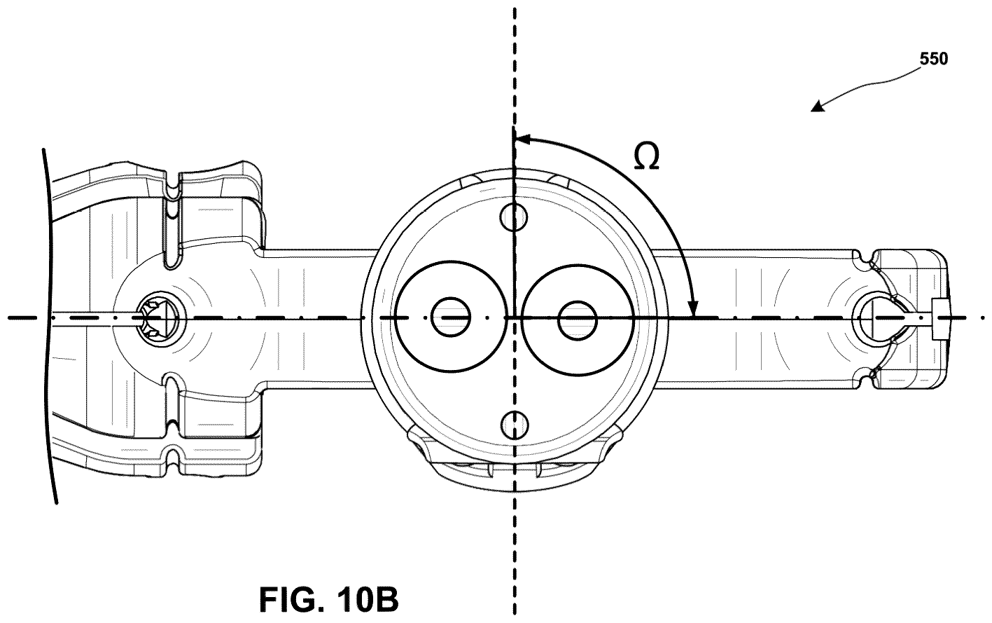

Referring now to FIGS. 10A and 10B, a further schematic example of a dual-deployment suture placement and injection device is shown that generally has the same aspects and features as the device of FIGS. 4A to 9B, except as shown and described herein. As depicted for the example arrangement of FIGS. 10A and 10B, in some arrangements, the first radial axis can define an acute angle with respect to the second radial axis, which is depicted as angle £2 in FIG. 10B. In some implementations, the first and second input openings for the channel guides can be defined through a top surface of the top portion of the housing; and the third and fourth openings for the input openings or ports for the injector channel guides can also defined through the top surface of the top portion. In addition, the first and second radial lines can be oriented normal to each other at the top surface as shown in FIG. 10B, but can also be oriented at another acute angle less than ninety degrees. Even though the first and second radial lines can have offset rotations at a top portion of the device and/or through a top surface thereof, both sets of channel guides can be routed moving distally from the top portion such that the pairs of channel guides are generally oriented parallel and adjacent one another through the device. Such an arrangement can be beneficial for dual-functionality deployment features of the device. However, the pairs of channel guides can also be routed and oriented independently of each through the device as appropriate in accordance with desired input port locations and/or injection positions at the distal end portion.

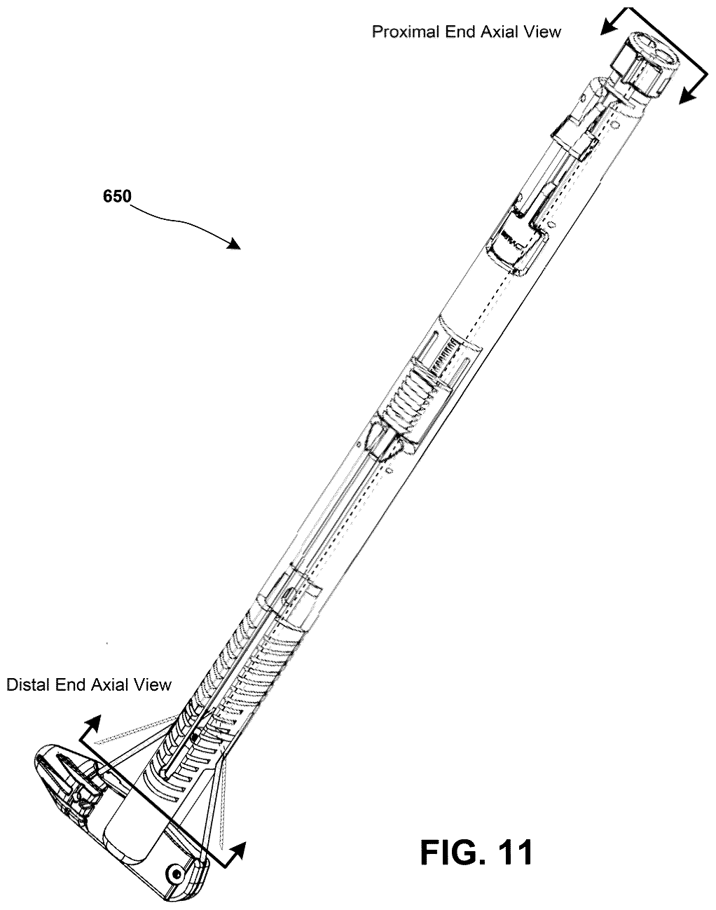

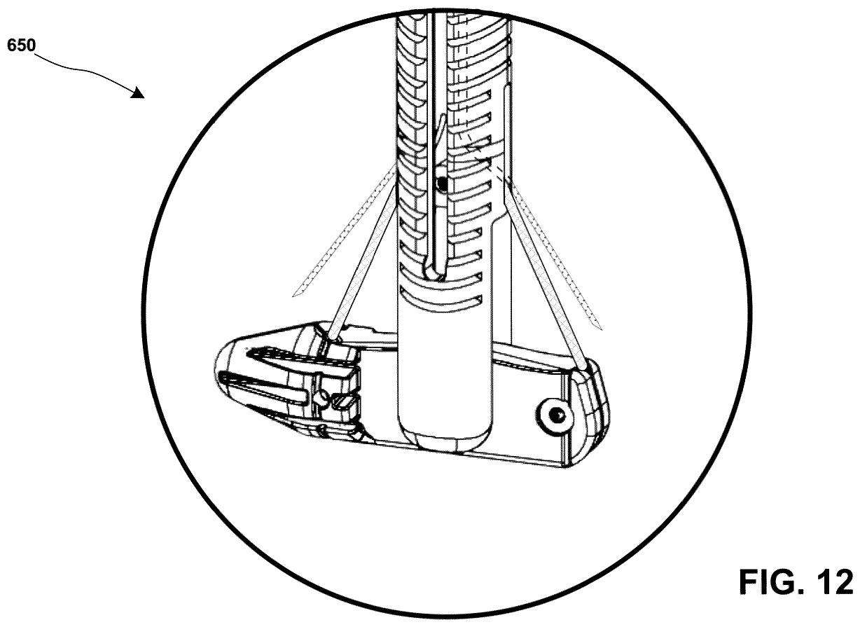

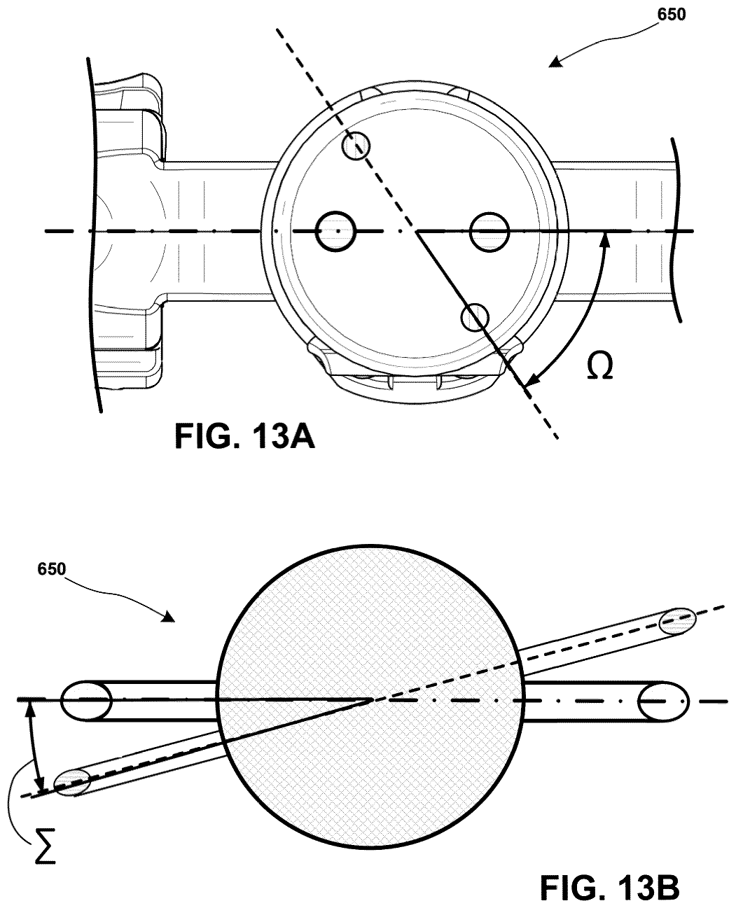

Referring now to FIGS. 12 to 13B, an additional schematic example of a dual-deployment suture placement and injection device is shown that generally has the same aspects and features as the device of FIGS. 4A to 11, except as shown and described herein. As shown in FIG. 12, in some arrangements the first and second channel guides or suture channel guides can each extend a first depth in the surgical port to form the channel pathway when in the respective deployed positions. Further, the third channel guide forming an injector channel guide can extend a second depth in the surgical port when in the deployed position at the first injection position, and the fourth channel guide forming another injector port can extend a third depth in the surgical port when in the deployed position at the second injection position. The third and fourth channel guides can be disposed at different heights from each other and can be oriented at different radial rotations from each other instead of oriented opposite one another. However, it can be beneficial for effective medicament delivery and dispersal including applications of local an anaesthesia for the opposing injector channel guides to be oriented and arranged radially opposite each other and at the same depth within the surgical port. In some arrangements as shown in FIG. 12, the second and third depths are less than the first depth and placed at a beneficial distance above distal ends of the suture channel guides, such as about about 0.1 to 1.0 cm proximal. In some arrangements, the first and the second injection positions and the second and third depths can be substantially the same.

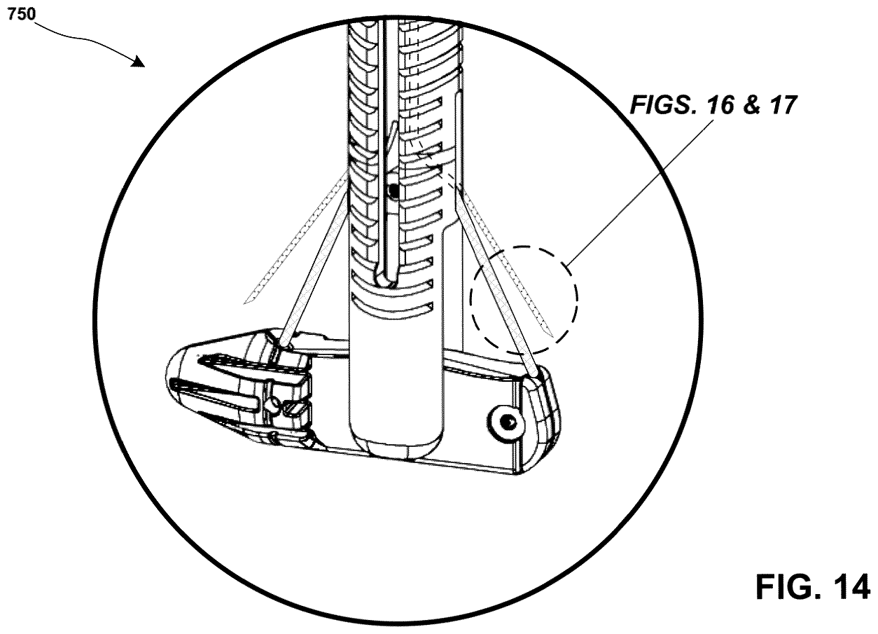

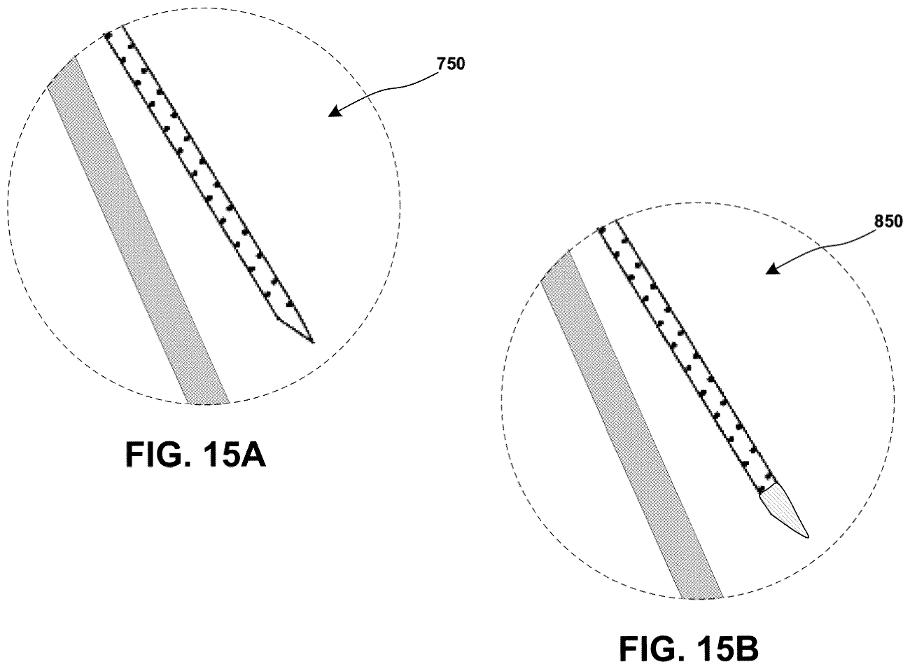

Referring now to FIGS. 14 and 15A, as well as FIG. 15B, an additional schematic example of a dual-deployment suture placement and injection device is shown in FIGS. 14 and 15A that generally has the same aspects and features as the device of FIGS. 4A to 13B, except as shown and described herein. As shown in FIGS. 14 and 15A, in some arrangements a portion of the injection path segment of each of the third and fourth channel guides or injector guide channels can be fenestrated. Such an arrangement can provide enhanced dispersal of medicaments injected and applied therethrough including local anaesthesia.

Referring now to FIG. 15B, which is an alternative example arrangement as a variation of the device of FIGS. 14 and 15A, an example dual-deployment suture placement and injection device is shown having a different example arrangement for the fenestrated end portion of one or more inject channel guides. As depicted in FIG. 15B, a distal end of at least one, and/or each of the third and fourth channel guides or injector channel guides can be substantially closed for blocking or throttling the flow of medicament through an axial opening at an end of the injector channel guide. In some implementations, the fenestrated portion of each injection path segment can be a distal end portion of the injection path segment, and a substantial portion of a deployed distal length of each injection path segment that extends from the housing can be fenestrated. Such an arrangement can limit excessive flow from the axial end and thereby provide enhanced dispersal of the medicament from the injector channel guide fenestration openings. Various fenestration parameters can be implemented for desired dispersal including opening sizes, density and length of openings along an end portion of the injector channel guides, surgical procedure and needs, injector tube inner diameter, injector tube material, and/or density of medicament to be injected. In some implementations, injector channel guides can be formed from flexible tubes, such as medical grade thermoplastic or elastomeric materials, as well as from flexible metals such as aluminium.

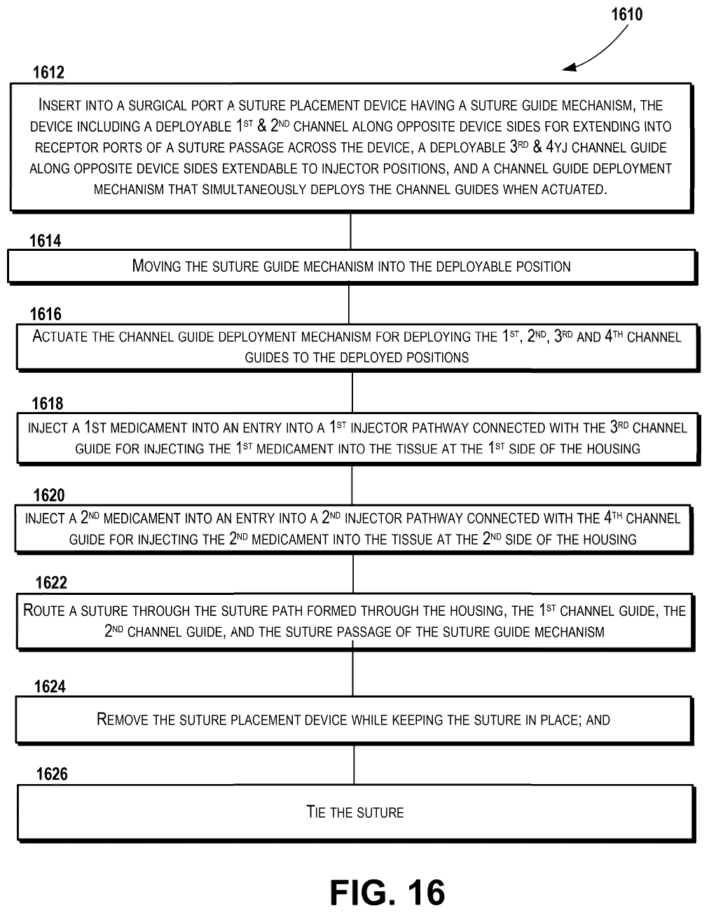

Referring now to FIG. 16, an example method for using a dual-deployment suture placement and injection device according to aspects and features described herein is generally shown as schematic representation. As depicted in FIG. 16, a method of use for a dual-deployment suture placement and injection device can include placing a suture and injecting a medicament in tissue through a surgical port therein having an outer diameter. The method can include inserting the dual-deployment suture placement and injection device into the surgical port, moving the suture guide mechanism into the deployed position, and actuating the channel guide deployment mechanism for simultaneously deploying the first, second, third and fourth channel guides to the deployed positions. Once deployed, the method can further include injecting a first medicament into an entry, such as an injector port of a first injector pathway connected with the third channel guide for injecting the first medicament into the tissue at the first side of the housing. In the deployed condition, the method can further include injecting a second medicament into an entry, such as an injector port, into a second injector pathway connected with the fourth channel guide for injecting the second medicament into the tissue at the second side of the housing. In some implementations, a medicament can be simultaneously injected into both the first and the second injector pathways via a injection adapter coupled to input ports for the third and fourth channel pathways. The method can further include routing a suture through a suture path formed through the housing, the first channel guide, the second channel guide and the suture passage of the suture guide mechanism, removing the dual-deployment suture placement and injection device while keeping the suture in place, and tying the suture.

In some methods, for injecting the first medicament, the first medicament can include at least one of an anaesthesia, a nsaid, and a therapeutic. Similarly, for injecting the second medicament, the second medicament can include at least one of an anaesthesia, a nsaid, and therapeutic. In some methods, for actuating the channel guide deployment mechanism, actuating the channel guide deployment mechanism can simultaneously deploy the first, second, third and fourth channel guides to the deployed positions. In some methods, for actuating the channel guide deployment mechanism, actuating the channel guide deployment mechanism can implement deployment of the first, second, third and fourth channel guides to the deployed positions in a sequential manner and/or combination of sequential and simultaneous, such as deploying a first pair of the channel guides followed by automatically deploying a second pair of the channel guides. Further, the method can include: providing an abdominal wall nerve block by deploying the third and the fourth channel guides to the deployed positions at the first and second injection positions spaced at desired distances of about 0.1 to 1.0 cm proximal from the first and second receptor ports, respectively, and injecting an anaesthetic into the third and fourth channel guides.

While this invention has been described as having exemplary designs, the present invention may be further modified within the spirit and scope of the disclosure. This application is therefore intended to cover any variations, uses, or adaptations of the invention using its general principles. Further this application is intended to cover such departures from the present disclosure as come within known or customary practice in the art to which this invention pertains.

We claim:

1. A dual-deployment suture placement and injection device comprising:
a cannular housing insertable into a surgical port of a determined width, the housing comprising:
a proximal portion, a distal portion, and longitudinal axis extending therebetween; and
a suture guide mechanism movably coupled to the distal portion of the cannular housing arranged for movement between a deployed position and a non-deployed position, the suture guide mechanism defining a first receptor port, a second receptor port, and a suture passage therebetween, the first and second receptor ports disposed on opposite side portions of the suture guide mechanism and the cannular housing in the deployed position wherein the suture passage extends across a width of the cannular housing in the deployed position;
a first channel guide within the housing comprising a proximal end, a distal end, and a guided suture path segment therebetween, the first channel guide movable between a retracted position oriented for passage through the surgical port and a deployed position where a portion of the first channel guide extends from the housing to the first receptor port;
a second channel guide within the housing comprising a proximal end, a distal end, and a guided suture path segment therebetween, the second channel guide movable between a retracted position oriented for passage through the surgical port and a deployed position wherein a portion of the second channel guide extends from the housing to the second receptor port;
a third channel guide within the housing comprising a proximal end, a distal end, and an injection path segment therebetween, the third channel guide movable between a retracted position oriented for passage through the surgical port and a deployed position wherein a portion of the third channel guide extends from a first side of the housing to a first injection position;
a fourth channel guide within the housing comprising a proximal end, a distal end, and an injection path segment therebetween, the fourth channel guide movable between a retracted position oriented for passage through the port and a deployed position wherein a portion of the fourth channel guide extends from a second side of the housing opposite from the first side to a second injection position; and
a channel guide deployment mechanism attached to the housing and each channel guide;
wherein actuation of the channel guide deployment mechanism deploys the first, second, third and fourth channel guides to each of the respective deployed positions.

2. The dual-deployment suture placement and injection device of claim 1, wherein actuation of the channel guide deployment mechanism automatically places the dual-deployment suture placement and injection device in condition for suturing and for injecting a medicament through the third and fourth channel guides.

3. The dual-deployment suture placement and injection device of claim 1, wherein, in the actuated and deployed configuration each of the first, second, third and fourth channel guides are in the respective deployed position:
a continuous suture pathway is defined through the dual-deployment suture placement and injection device from a first opening at the proximal portion of the housing through each of the first and second channel guides and the suture guide mechanism therebetween to a second opening at the proximal portion of the housing;
a first injection pathway is defined through the dual-deployment suture placement and injection device from a third opening at the proximal portion of the housing through the third channel guide to the distal end of the third stylet guide at the first injection position; and
a second injection pathway is defined through the dual-deployment suture placement and injection device from a fourth opening at the proximal portion of the housing, through the fourth channel guide to the distal end of the fourth channel guide at the second injection position.

4. The suture placement device of claim 3, wherein:
the first opening at the proximal portion of the housing and the second opening at the proximal portion of the housing are formed through a top portion of the housing, the first and second openings axially aligned with each other across a first radial axis of the housing top portion; and
the third opening at the proximal portion of the housing and the fourth opening at the proximal portion of the housing are formed through a top portion of the housing, the third and the fourth openings axially aligned with each other across a second radial axis of the housing top portion.

5. The suture placement device of claim 4, wherein:
the first radial axis and the second radial axis are generally aligned with each other such that the first, second, third and fourth openings are generally radially aligned along the same radial axis across the housing top portion.

6. The suture placement device of claim 4, wherein:
the first radial axis defines an acute angle with the second radial axis.

7. The suture placement device of claim 4, wherein:
the first and second openings are defined through a top surface of the top portion of the housing;
the third and fourth openings are defined through a proximal side portions of the housing at the top portion of the housing; and
the suture placement device further comprises an injection connector configured for coupling each of the third and fourth openings with a top portion of a medicament injector.

8. The dual-deployment suture placement and injection device of claim 1, wherein:
the first and second channel guides each extend a first depth in the surgical port when in the respective deployed positions;
the third channel guide extends a second depth in the port when in the deployed position at the first injection position;
the fourth channel guide extends a third depth in the port when in the deployed position at the second injection position; and
the second and third depths are less than the first depth.

9. The dual-deployment suture placement and injection device of claim 8, wherein the first and the second injection positions and the second and third depths are substantially the same.

10. The dual-deployment suture placement and injection device of claim 1, wherein in the actuated and deployed configuration each of the first, second, third and fourth channel guides are in the respective deployed position:
the first channel guide and the third channel guide are disposed proximate each other extending from a first side of the housing; and
the second channel guide and the fourth channel guide are disposed proximate each other extending from a second side of the housing opposite from the first side of the housing.

11. The dual-deployment suture placement and injection device of claim 1, wherein in the actuated and deployed configuration each of the first, second, third and fourth channel guides are in the respective deployed position:
the first channel guide extends from a third side of the housing;
the second channel guide extends from a fourth side of the housing opposite from the third side of the housing, the first and second channel guides defining a first radial axis therebetween;
the third channel guide and the fourth channel guide define a second radial axis therebetween; and
an acute angle is defined between the first and the second radial axes.

12. The dual-deployment suture placement and injection device of claim 11, wherein the third channel guide and the fourth channel guide are oriented substantially perpendicular to the first channel guide and the second channel guide, and the acute angle is about ninety degrees.

13. The dual-deployment suture placement and injection device of claim 1, wherein:
a portion of the injection path segment of each of the third and fourth channel guides is fenestrated; and
the distal end of each of the third and fourth channel guides is substantially closed.

14. The dual-deployment suture placement and injection device of claim 13, wherein the fenestrated portion of each injection path segment is a distal end portion of the injection path segment.

15. The dual-deployment suture placement and injection device of claim 13, wherein a substantial portion of a deployed distal length of each injection path segment that extends from the housing is fenestrated.

16. The dual-deployment suture placement and injection device of claim 1, wherein the third channel guide and the fourth channel guides are flexible tubes.

17. A method of placing a suture and injecting a medicament in tissue through a surgical port therein having an outer diameter, the method comprising:
inserting a dual-deployment suture placement and injection device as recited in claim 1 into the surgical port;
moving the suture guide mechanism into the deployed position;
actuating the channel guide deployment mechanism for deploying the first, second, third and fourth channel guides to the deployed positions;
injecting a first medicament into an entry into a first injector pathway connected with the third channel guide for injecting the first medicament into the tissue at the first side of the housing;
injecting a second medicament into an entry into a second injector pathway connected with the fourth channel guide for injecting the second medicament into the tissue at the second side of the housing;
routing a suture through a suture path formed through the housing, the first channel guide, the second channel guide and the suture passage of the suture guide mechanism;
removing the dual-deployment suture placement and injection device while keeping the suture in place; and
tying the suture.

18. The method of claim 17, wherein:
for injecting the first medicament, the first medicament comprises at least one of an anesthesia, a NSAID, and therapeutic; and
for injecting the second medicament, the second medicament comprises at least one of an anesthesia, a NSAID, and therapeutic.

19. The method of claim 17, wherein, for actuating the channel guide deployment mechanism, actuating the channel guide deployment mechanism simultaneously deploys the first, second, third and fourth channel guides to the deployed positions.

20. The method of claim 17, further comprising:
providing an abdominal wall nerve block by deploying the third and the fourth channel guides to the deployed positions at the first and second injection positions spaced at desired distances of about 0.1 to 1.0 cm proximal from the first and second receptor ports, respectively, and injecting an anesthetic into the third and fourth channel guides.
